(19) United States
(12) Patent Application Publication
(10) Pub. No.: US 2005/0191705 A1
(43) Pub. Date: Sep. 1, 2005

(54) High throughput screening using fluorophore labeled lipid membranes and fluorescence correlation spectroscopy

(76) Inventors: James H. Werner, Los Alamos, NM (US); Scott M. Reed, Portland, OR (US); Basil I. Swanson, Los Alamos, NM (US)

Correspondence Address: UNIVERSITY OF CALIFORNIA;LOS ALAMOS NATIONAL LABORATORY, P.O. BOX 1663, MS A187, LOS ALAMOS, NM, 87545, US

(21) Appl. No.: 10/790,638

(22) Filed: Mar. 1, 2004

(51) Int. Cl. G01N 33/53

(52) U.S. Cl. 435/7.1

(57) ABSTRACT

An apparatus for and method of detecting a binding event between biomolecules is disclosed and includes admixing a target molecule including a first fluorophore and membrane vesicles including a trifunctional linker molecule, said trifunctional linker molecule including a second fluorophore, to form a sample, introducing a library of elements into said sample, each of said library elements having a binding affinity for said trifunctional linker molecule, and, screening said sample for fluorescence from said first fluorophore and said second fluorophore, such fluorescence indicative of a binding event between an element from said library of elements and said target molecule.

### STATEMENT REGARDING FEDERAL RIGHTS

This invention was made with government support under Contract No. W-7405-ENG-36 awarded by the U.S. Department of Energy. The government has certain rights in the invention.

### FIELD OF THE INVENTION

The present invention relates to fluorescence-based assays to detect and monitor interactions between biochemical molecules. More particularly, the invention relates to an apparatus for and method of detecting binding between biochemical molecules by fluorescence correlation spectroscopy using fluorophore-labeled lipid membranes. Such a method and apparatus provide a means of rapidly screening large, combinatorial libraries to discover and quantify binding interactions.

### BACKGROUND OF THE INVENTION

Various screening techniques have been developed to detect and monitor interactions between biochemical molecules and to identify biochemical molecules with unique features such as binding, inhibiting or catalytic functions. High-throughput screening is a process in which batches of compounds (e.g., molecular library elements) are tested for binding activity or biological activity against target molecules. The potential market for application of biosensor or high-throughput screening technologies is enormous and includes detection and diagnostics in the health care industry and environmental monitoring.

Screening techniques are generally based on detecting and monitoring a binding event between a recognition and target molecule. These molecules can be peptides, antibodies, antibody fragments, receptors, oligonucleotides, and oligosaccharides. These binding events include binding of a target molecule at a single binding site on a recognition molecule as well as binding at multiple sites (i.e., multivalent binding) of a target molecule by multiple recognition molecules.

Screening techniques incorporate a variety of detection methods. One highly sensitive detection technique is fluorescence correlation spectroscopy (FCS). FCS measures fluctuations in fluorescence intensity from a small number of fluorescently tagged molecules diffusing through a small detection volume (typically less than about 1 femtoliter) over a defined time range (typically microseconds to seconds). Diffusion of the tagged molecule through the detection volume produces a fluctuation in fluorescence intensity that is detected and discriminated from background noise by auto- or cross-correlation. The correlation function includes quantitative information about concentration and diffusion rates (e.g., molecular mass) of molecules in the sample. For example, the average time required for passage of a single fluorescent molecule through the detection volume is determined by its diffusion coefficient, which is related to the size of the molecule. Small, rapidly diffusing molecules produce rapidly fluctuating intensity patterns, compared with larger molecules that produce more sustained patterns of fluorescence.

FCS has been used in high-throughput screening assays to detect binding between a recognition molecule (e.g., a library element, such as an antibody) and a target molecule (e.g., an antigen). For example, a target molecule, such as an antigen with different binding epitopes can be used to screen a fluorescently labeled antibody library (e.g., a red fluorophore) for antibodies that recognize and bind the different antigen epitopes. Diffusion of the tagged molecule through detection volume produces a fluctuation in fluorescence intensity that can be detected and discriminated from background noise by auto correlation. Binding of a labeled antibody to the antigen can be detected by a shift from a rapidly fluctuating red fluorescence pattern to a more sustained or prolonged pattern of red fluorescence. However, the target molecule and recognition molecule must be sufficiently different in molecular mass, generally by about a factor of 10, in order for a shift in the fluorescence pattern upon binding to be detected. Thus, a need exists for a FCS screening assay that simultaneously labels a library element with a fluorescent tag and significantly increases its effective size. Such a method is now provided by the present invention.

Another limitation in current high-throughput screening assays is that the binding between a target molecule and a recognition molecule often occurs at a single recognition site. Single-site binding events are often associated with issues such as low binding affinities and high on-off rates, and consequently the binding event is less stable and harder to detect. Multivalent binding events are generally more stable because they include multiple sites of interaction between a target molecule and recognition molecules. Multivalent binding events overcome the instability issues associated with single-site binding events, and are therefore easier to detect. This, a need exists for a FCS screening assay that is based on binding at multiple sites (i.e., multivalent binding) of a target molecule by multiple recognition molecules.

A final limitation of current screening methodologies lies in the fact that several protein receptors and oligosaccharides are water-insoluble and in nature are found segregated into the cellular membrane. The present invention enables the study of these water-insoluble molecules by surrounding such species with an appropriate lipophilic, biomimetric surrounding, i.e., a vesicle.

### SUMMARY OF THE INVENTION

To achieve the foregoing and other objects, and in accordance with the purposes of the present invention, as embodied and broadly described herein, the present invention provides an apparatus including a light source, an objective, a first detector means for detecting light of a first defined wavelength range, a second detector means for detecting light of a second defined wavelength range, a first filter means for filtering light of a third defined wavelength range, a second filter means for filtering light of a fourth defined wavelength range, a support having a pinhole therein through which collected light from said objective is preferentially passed to said first detector means and said second detector means as opposed to out of focus scattered light, and, a transparent substrate for support of a sample under investigation, said sample comprising membrane vesicles including a trifunctional linker molecule including a fluorophore.

The present invention further provides a method of detecting a binding event between biomolecules including admixing a target molecule including a first fluorophore and membrane vesicles including a trifunctional linker molecule, said trifunctional linker molecule including a second fluorophore, to form a sample, introducing a library of elements into said sample, each of said library elements having a binding affinity for said trifunctional linker molecule, and, screening said sample for fluorescence from said first fluorophore and said second fluorophore, such fluorescence indicative of a binding event between an element from said library of elements and said target molecule.

One embodiment of the present method involves use of fluorescence cross correlation to screen samples for a binding event by checking for correlations in the fluorescence light intensity measured by spectrally resolved detectors.

Another embodiment of the present method involves use of fluorescence correlation spectroscopy to screen samples for a binding event by examination of temporal durations that result from diffusion coefficients by target molecules bound to membrane vesicles.

### BRIEF DESCRIPTION OF THE DRAWINGS

FIG. 1 illustrates an example of a trifunctional linker molecule including a membrane anchoring group, a reporter group and a reactive or recognition group.

FIG. 2(a) and 2(b) illustrate a schematic representation of a vesicle-based detection system.

FIG. 3 illustrates a schematic representation of a detection apparatus for measurement of a sample using FCS.

FIG. 4 illustrates a method of using the vesicle-based detection system in conjunction with the detection apparatus.

FIG. 5(a) illustrates autocorrelation functions of unbound target and bound target and FIG. 5(b) illustrates cross-correlation functions in the presence and absence of a binding event between two molecules labeled with spectrally distinct fluorophores.

### DETAILED DESCRIPTION

The present invention concerns a method of monitoring and screening molecular interactions. More particularly, the invention concerns a method of monitoring (e.g., chemical coupling reactions) and screening (e.g., display and combinatorial libraries) a sample by FCS using fluorophore-labeled lipid membranes.

The present invention provides a method to detect and to quantify binding events with target molecules. Such a method can be useful in the development of new pharmaceutical, therapeutic and sensing molecules. The method allows fast analysis times and miniscule sample requirements and can serve as a valuable tool in the screening of large combinatorial libraries for biological and chemical applications.

A preferred embodiment of the present invention involves use of a trifunctional linker as described in U.S. Pat. No. 6,627,396, such a trifunctional linker including alkyl chain groups for anchoring or attachment to a substrate such as a lipid membrane substrate, a fluorescent moiety capable of generating a fluorescent signal, and a recognition moiety with a spacer group of a defined length, the recognition moiety for binding with a target molecule.

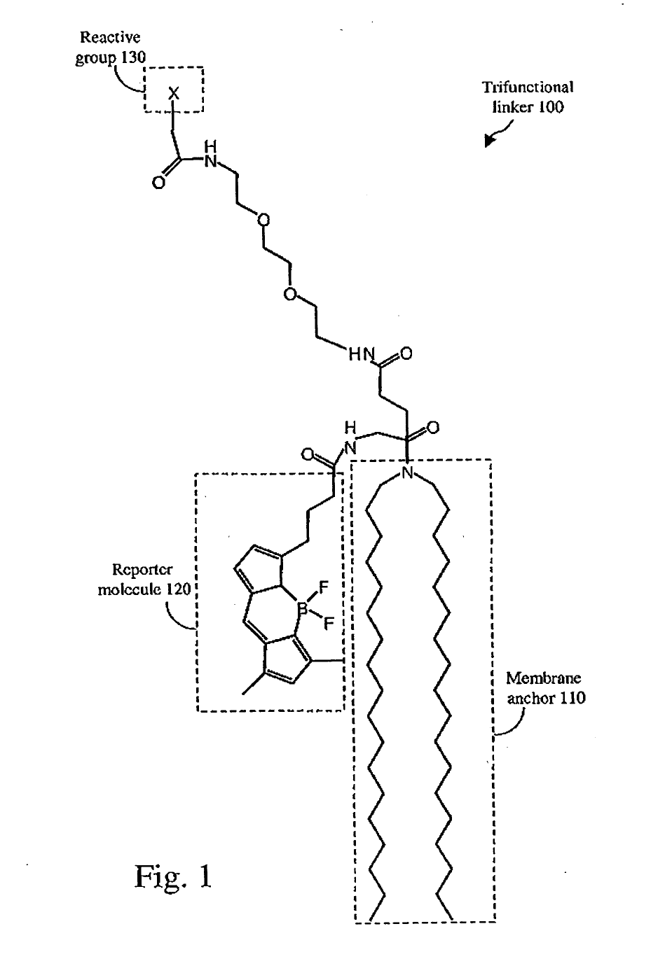

FIG. 1 illustrates an example of a trifunctional linker molecule 100 including a membrane anchoring group 110, a reporter group 120 and a reactive or recognition group 130.

Reactive group 130 (or alternatively, recognition group 130) provides a chemically reactive site for coupling a recognition molecule, such as a peptide, to trifunctional linker molecule 100. Other recognition molecules are well known by those skilled in the art and can be used as well.

Reporter group 120 can typically be any chemical or biochemical entity or label that yields an externally measurable output signal that can be correlated or assigned with a specific binding event. Suitable examples of such groups can include fluorophores, isotopic labels or magnetic materials. Suitable fluorophores can be from the group of rhodamines, an intrinsically fluorescent protein, such as green fluorescent proteins (GFP), fluoresceins and the like.

Membrane anchoring group 110 provides mobile attachment of the entire trifunctional linker molecule 100 to a fluid surface of a membrane. Such a membrane anchoring group 110 can generally be any group that contains alkyl, alkenyl, alkynyl, and polyaromatic chains of carbon atoms containing from about 4 to about 30 carbon atoms. One preferred anchoring groups are long chain alkyl groups such as straight chain alkyl groups with 18 carbon atoms.

Trifunctional linker molecule 100 can be inserted into a lipid membrane, typically by adding a solution containing the linker molecule directly to a membrane lipid solution used to form vesicles. Trifunctional linker molecule 100 is incorporated into vesicles or micelles with reactive group 130 exposed on both the external and internal vesicle surfaces. Standard conjugation chemistry can be used to covalently attach a recognition molecule to reactive group 130.

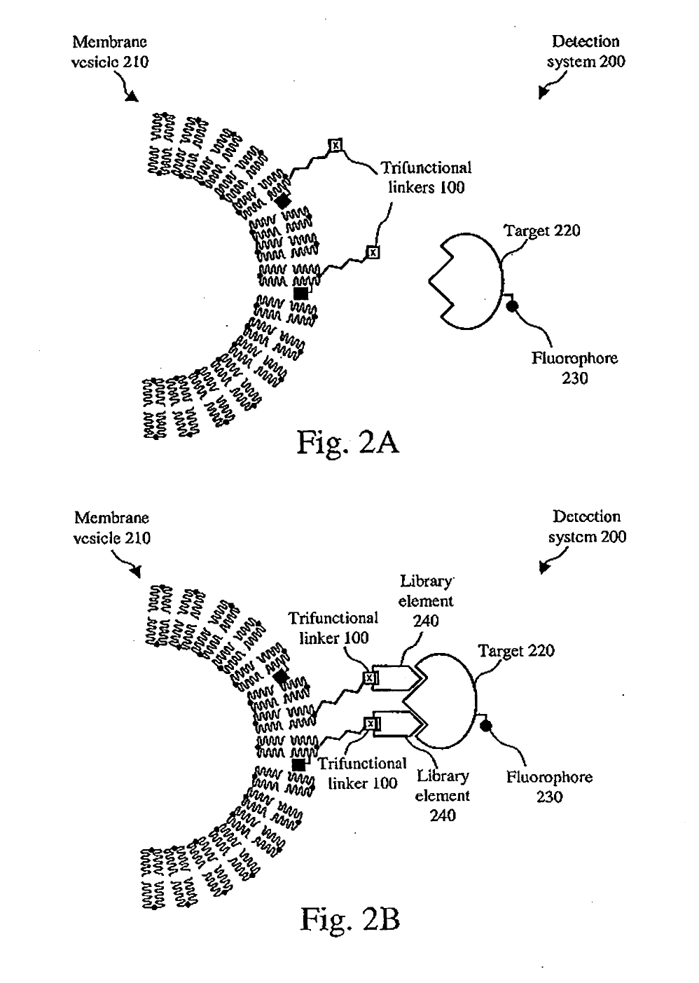

FIG. 2(a) illustrates a schematic representation of a vesicle-based detection system 200. In one embodiment of the present invention, detection system 200 includes a membrane vesicle 210 and a target 220. Target 220 is typically significantly smaller (i.e., an order of magnitude) than membrane vesicle 210. Protein targets typically weigh from about 10,000 daltons to about 100,000 daltons and are approximately 10 nanometers (nm) in diameter. Suitable vesicles can then typically be 100 nm in diameter or larger. Larger sizes of vesicles can enhance the contrast in correlation time.

In this embodiment, target 220 further includes a fluorophore 230. Target 220 is any target molecule of interest, such as a peptide or the like. In the presently illustrated embodiment, target 220 is a multivalent molecule. In an alternative embodiment, target 220 can have a single binding site.

Fluorophore 230 can be any fluorescent molecule that is distinct from reporter molecule 120 attached to trifunctional linker 100. For example, fluorophore 230 could be a green fluorescent molecule where reporter molecule 120 is a red fluorescent molecule.

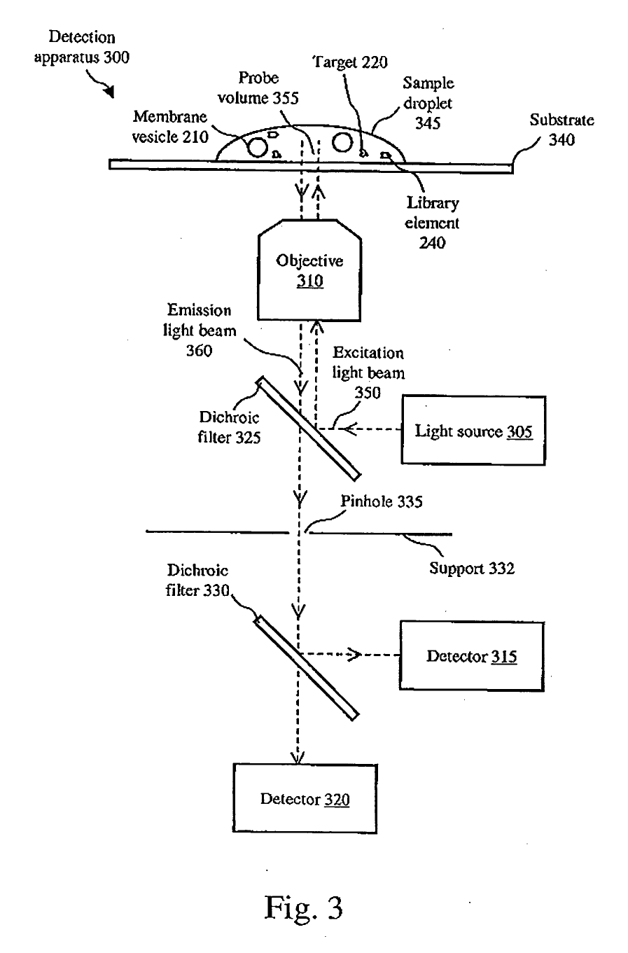

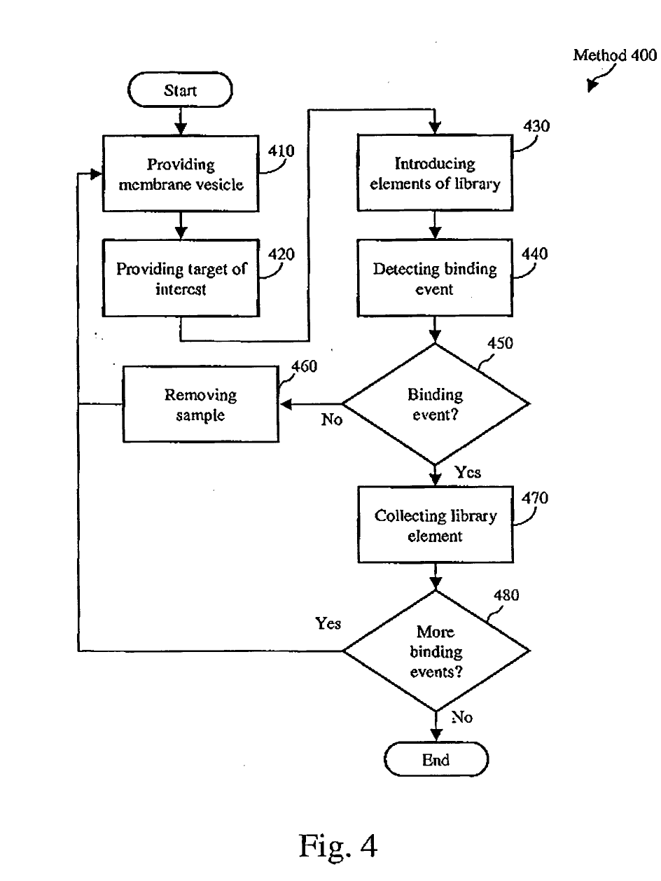

The binding of target 220 by library element 240 can be detected and analyzed using FCS as described in reference to FIG. 3 and FIG. 4. FIG. 3 illustrates a schematic representation of a detection apparatus 300 for FCS measurement of a sample. Detection apparatus 300 includes a light source 305, an objective 310, a first detector means 315, a second detector means 320, a first filter means 325, a second filter means 330, a support 332 having a pinhole 335 therein, and a substrate 340. In operation, detection apparatus 300 further includes an aqueous sample droplet 345, an excitation light beam 350, a probe volume 355, and an emission light beam 360. Detection apparatus 300 can typically be an epifluorescence detection system, in which excitation light beam 350 travels through objective 310 to illuminate sample droplet 345 deposited upon substrate 340. Substrate 340 can be any transparent substrate, such as a glass microscope slide or slipcover, which facilitates transmission of both excitation light beam 350 and emission light beam 360. Emission light beam 360 from sample droplet 345 is subsequently collected and focused by objective 310. Sample droplet 345 further includes a plurality of membrane vesicles 210, targets 220, and library elements 240.

Light source 305 can be any conventional light source, such as a specific wavelength laser or a mercury vapor arc burner, which provides excitation light beam 350 suitable for the excitation of fluorophores within membrane vesicles 210 and target 220.

Objective 310 can be any conventional converging lens, such as a 60× Nikon CFN plan apochromat, which focuses and transmits light. Probe volume 355 is the area of penetration of excitation light beam 350 from objective 310, and represents the area of sample droplet 345 under FCS analysis.

Detector means 315 and detector means 320 can be conventional optical sensors, such as avalanche photodiodes (SPCM 200 PQ, from Perkin Elmer Optoelectronics, Quebec, Canada) for detecting light of a specific wavelength. In the presently described embodiment, those wavelengths would be for green and red light, respectively.

Filter means 325 and 330 can be dichroic filters, e.g., conventional longpass optical filters, such as XF2010 (Omega Optical), that reflect light shorter than a certain wavelength, and pass light longer than that certain wavelength. For example, filter means 325 can be a filter that reflects wavelengths below 500 nm (where the wavelength of excitation beam 350 is at 496 nm). This filter then passes light above 500 nm, where fluorescence emission 360 occurs, i.e., the emitted fluorescence 310. The emission light beam 360 can be further spectrally filtered by detector means 330. This filter then reflects emission light beam 360 below 550 nm, and passes emission light beam 360 above 550 nm from sample droplet 345.

Pinhole 335 formed within support 332 acts as a spatial filter to block scattered laser light and penetration of “out of focus” emission light beam 360 from sample droplet 345 through objective 310. For example, “out of focus” emission light beam 360 is typically light that is not at the focal point of objective 310. Pinhole 335 effectively provides penetration of “in focus” emission light beam 360 to detector means 315 and detector means 320 via filter means 330.

FIG. 4 illustrates a method 400 of using detection system 200 in conjunction with detection apparatus 300 according to a preferred embodiment of the present invention. The preferred embodiment includes the use of two different fluorophores, i.e., a first fluorophore as a structural component of trifunctional linker 100 anchored in membrane vesicles 210 and a second fluorophore as part of targets 220. Cross-correlation analysis of the two different fluorophores decreases background fluorescence from unbound membrane vesicles 210 and targets 220, and increases the sensitivity of detecting a binding event. Method 400 generally includes the first step of providing membrane vesicles 410, as previously described in reference to FIGS. 2(a) and 2(b). The next step 420 is that of providing a target of interest. In step 420, a target of interest 220 is provided. For example, target 220 can be added using standard microfluidic techniques to an aqueous solution containing membrane vesicles 210 to form sample droplet 345. Sample droplet 345 is typically about 4 microliters in volume. The next step 430 is that of introducing elements of a library. In step 430, elements of a library (such as library element 240) are provided. Library element 240 is introduced into sample interrogation region 345, typically by standard microfluidic techniques, such as a stop/flow mechanism. Sample interrogation region 345 now includes membrane vesicles 210, target 220, and library element 240.

The next step 440 is detecting a binding event. In step 440, fluorescence detection is performed to detect a binding event. In a preferred embodiment, fluorescence detection is performed by FCS using detection apparatus 300 as described above. FCS is a standard technique commonly used in fluorescence-based detection assays.

In a typical FCS measurement, (i.e., autocorrelation or cross-correlation), fluorescence intensity is recorded over a time range from seconds to minutes. The time-dependent fluorescence intensity (I(t)) is then analyzed in terms of its temporal correlation function (G(τ)), which compares the fluorescence intensity at time t with the intensity at (t+τ), where τ is a variable interval averaged over all data points in a time series. Mathematical auto- or cross-correlation of the data uses the following general formula:
G(τ)=<δI₁(t)δI₂(t+τ)>/<I₁(t)><I₂(t)>

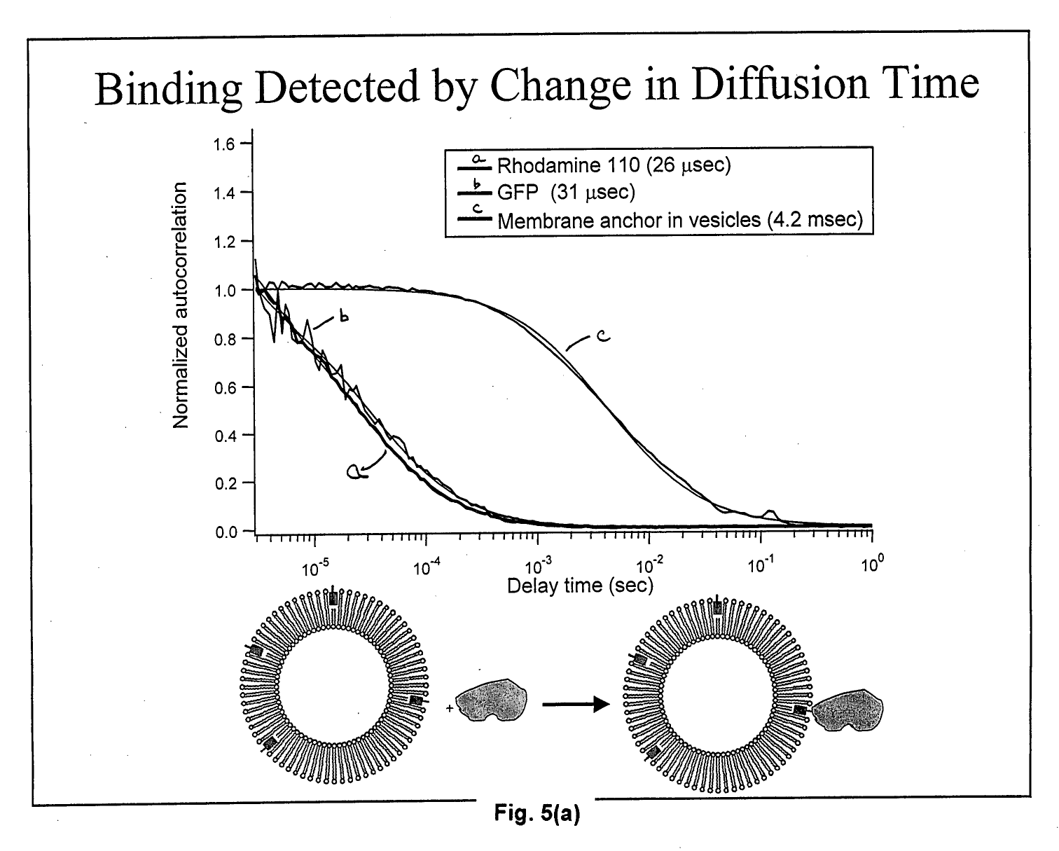

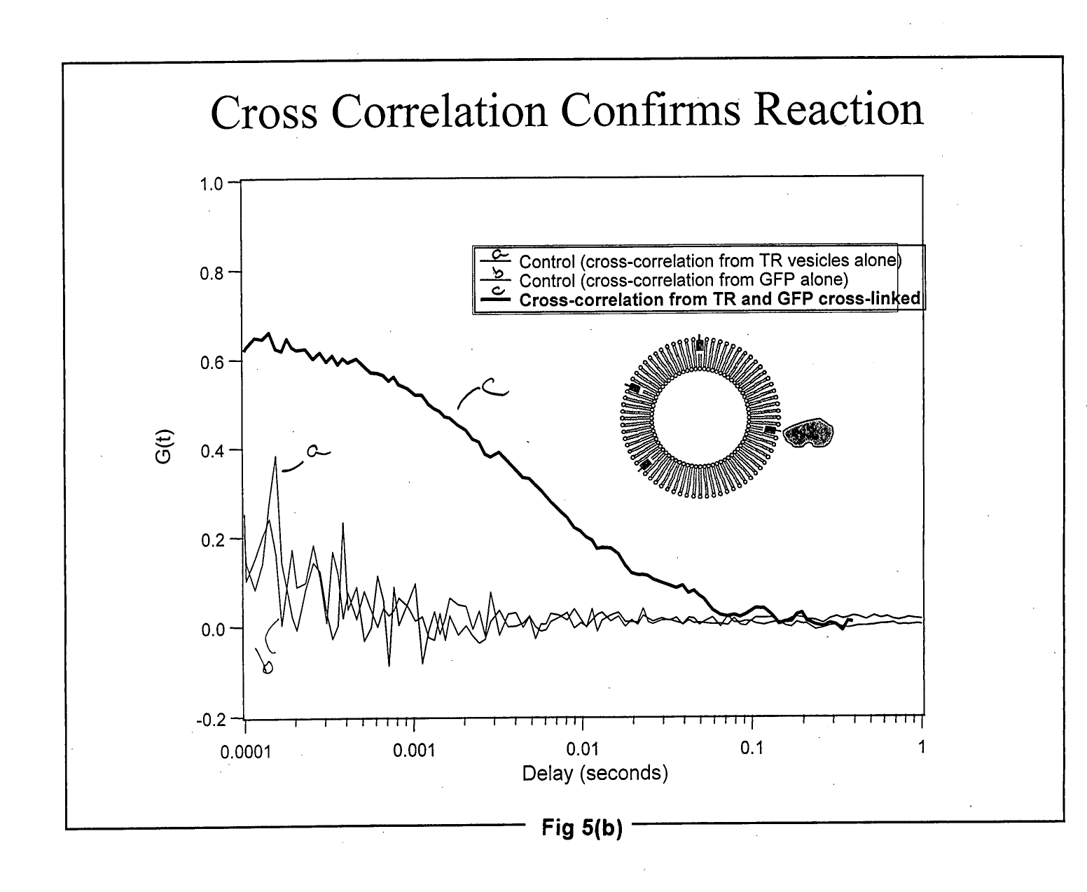

The autocorrelation function measures the time-dependent fluorescence intensity (I(t)) for a single fluorophore where I₁ and I₂ are fluorescence intensity signals at different delay times. The autocorrelation function provides quantitative data on the concentration and size (i.e., diffusion rates) of molecules in a sample. The autocorrelation function further provides information on the interaction of two different molecules based on their differences in diffusion characteristics, as is shown and described further in FIG. 5(a).

The cross-correlation function measures the time-dependent fluorescence intensities of two spectrally distinct fluorophores where I₁ and I₂ are fluorescence intensity signals for different wavelengths, e.g., a green fluorescent signal and a red fluorescent signal. The cross-correlation function provides quantitative information on the specific interactions between two molecules labeled with the spectrally distinct fluorophores. A cross-correlation signal is generated only when the two distinct fluorophores are detected in a single binding complex, as is shown and described further in FIG. 5(b). Cross-correlation analysis eliminates background fluorescence from non-interacting molecules and increases the sensitivity of detecting a binding event.

In a preferred embodiment, two different fluorophores are used to detect a binding event. The time-dependent fluorescence intensity of one fluorophore, such as a green fluorescent signal, and the time-dependent fluorescence intensity of a second fluorophore, such as a red fluorescent signal, are cross-correlated to determine whether the two fluorescent signals occur in the same binding event, i.e., whether they are co-localized to a single molecular complex.

In operation, sample droplet 345 is excited by excitation light beam 350 from light source 305. Excitation light beam 350 is of sufficient wavelength to excite reporter molecule 120 (e.g., a red fluorophore) anchored in membrane vesicle 210 and fluorophore 230 (e.g., a green fluorophore) attached to target 220. The movement via random diffusion of membrane vesicle 210 and target 220 into and out of probe volume 355 is detected by detector means 320 and detector means 315, respectively. The time-dependent fluorescence intensity (I(t)) of each fluorophore is then analyzed in terms of its temporal correlation function (G(τ)), as described above.

The next step 450 is the determination whether a positive binding event has occurred. If the determination of whether a positive binding event has occurred is yes, then the process proceeds to step 470. If the determination is no, then the process proceeds to step 460.

In step 460, sample droplet 345 is removed from substrate, typically by standard microfluidic techniques, such as a stop/flow mechanism. Sample droplet 345 can then be discarded and the process returned to the beginning with step 410.

In step 470, sample droplet 345 is removed from substrate, typically by standard microfluidic techniques, such as a stop/flow mechanism. Sample droplet 345 is then stored for isolation and analysis of the particular library element 240. In one embodiment, the output is collected in the capillary of a tube. In an array embodiment, the droplet could be suctioned off with a pipette or capillary for further analysis. A next step 480 could then be used to examine for additional binding events.

FIG. 5(a) illustrates autocorrelation functions of unbound target 220 and bound target 220. The autocorrelation function measures the time-dependent fluorescence intensity for a single fluorophore (e.g., only target 220 is fluorescently labeled) and provides information on the interaction of two different molecules based on differences in their diffusion characteristics. Detection of a binding event between target 220 and a recognition molecule, such as an antibody, requires that the interacting components differ in molecular mass by at least a factor of about 10. The initial amplitude of the autocorrelation function is inversely proportional to the number of targets 220 in probe volume 355. The autocorrelation function decays from its initial value with a time-dependence that is determined by the molecular diffusion rates of target 220. Target 220, typically a lower molecular weight molecule, exhibits a faster autocorrelation decay in its unbound state (approximately 0.1 milliseconds) and a slower autocorrelation decay (about 5 milliseconds) when bound to a membrane vesicle.

FIG. 5(b) illustrates cross-correlation functions in the presence and absence of a binding event between two molecules labeled with spectrally distinct fluorophores. For example, target 220 is labeled with a green fluorophore such as a green fluorescent protein (GFP) and membrane vesicle 210 is labeled with a red fluorophore such as Texas Red. In the absence of a binding event between target 220 and membrane vesicle 210, no cross-correlation signal is generated. A cross-correlation signal is generated only when the two distinct fluorophores are detected in a single binding complex, i.e., target 220 is bound to membrane vesicle 210. The cross-correlation signal is independent of diffusion rates (i.e., molecular mass) of the interacting molecules.

Although the present invention has been described with reference to specific details, it is not intended that such details should be regarded as limitations upon the scope of the invention, except as and to the extent that they are included in the accompanying claims.

1. An apparatus comprising:
a light source;
an objective;
a first detector means for detecting light of a first defined wavelength range;
a second detector means for detecting light of a second defined wavelength range;
a first filter means for filtering light of a third defined wavelength range;
a second filter means for filtering light of a fourth defined wavelength range;
a support having a pinhole therein through which collected light from said objective is preferentially passed to said first detector means and said second detector means as opposed to out of focus scattered light; and,
a transparent substrate for support of a sample under investigation, said sample comprising membrane vesicles including a trifunctional linker molecule including a fluorophore.

2. The apparatus of claim 1 wherein said objective is a converging lens.

3. The apparatus of claim 1 wherein said first filter means and said second filter means are dichroic mirrors.

4. The apparatus of claim 1 wherein said first filter means is a longpass optical filter reflecting excitation wavelengths and passing fluorescence emission wavelengths and said second filter means spectrally resolves said fluorescence emission wavelengths.

5. The apparatus of claim 3 wherein said first dichroic mirror reflects wavelengths below 500 nm and passes wavelengths above 500 nm and said second dichroic mirror reflects wavelengths below 550 nm and passes wavelengths above 550 nm.

6. The apparatus of claim 1 wherein said transparent substrate is of glass.

7. The apparatus of claim 1 wherein said apparatus is characterized as having a single detection channel.

8. A method of detecting a binding event between biomolecules comprising:
admixing a target molecule including a first fluorophore and membrane vesicles including a trifunctional linker molecule, said trifunctional linker molecule including a second fluorophore, to form a sample;
introducing a library of elements into said sample, each of said library elements having a binding affinity for said trifunctional linker molecule; and,
screening said sample for fluorescence from said first fluorophore and said second fluorophore, such fluorescence indicative of a binding event between an element from said library of elements and said target molecule.

9. The method of claim 8 wherein said screening of said sample for a binding event includes monitoring for correlations in the fluorescence light intensity measured by spectrally resolved detectors.

10. The method of claim 8 wherein said screening of said sample for a binding event includes monitoring for temporal durations that result from diffusion coefficients by target molecules bound to said membrane vesicles.

11. The method of claim 8 wherein said first fluorophore is a green fluorophore and said second fluorophore is a red fluorophore.

12. The method of claim 8 wherein said second fluorophore is a red fluorophore.

13. A method of detecting a binding event between biomolecules comprising:
admixing a target molecule including a first fluorophore and membrane vesicles including a trifunctional linker molecule to form a sample, said membrane vesicles including a second fluorophore selected from the group of amphiphilic fluorophores or dye molecules encapsulated within said membrane vesicles;
introducing a library of elements into said sample, each of said library elements having a binding affinity for said trifunctional linker molecule; and,
screening said sample for fluorescence from said first fluorophore and said second fluorophore, such fluorescence indicative of a binding event between an element from said library of elements and said target molecule.

14. The method of claim 13 wherein said screening of said sample for a binding event includes monitoring for correlations in the fluorescence light intensity measured by spectrally resolved detectors.

15. The method of claim 13 wherein said screening of said sample for a binding event includes monitoring for temporal durations that result from diffusion coefficients by target molecules bound to said membrane vesicles.
